(19) United States
(12) Patent Application Publication
(10) Pub. No.: US 2026/0061113 A1
(43) Pub. Date: Mar. 5, 2026

(54) EXTENDABLE VAGINAL AND ANAL APPLICATOR

(71) Applicant: Moxikom, LLC, Oldsmar, FL (US)

(72) Inventors: Kimberly Moody, Oldsmar, FL (US); Bridget Conzemius, Oldsmar, FL (US)

(73) Assignee: Moxikom, LLC, Oldsmar, FL (US)

(21) Appl. No.: 18/823,844

(22) Filed: Sep. 4, 2024

(51) Int. Cl. A61M 3/02

(52) U.S. Cl. CPC A61M 3/0279; A61M 3/0262; A61M 2210/1067; A61M 2210/1475

(57) ABSTRACT

A vaginal and anal applicator apparatus that has a squeezable fluid storage bottle and a telescoping tube which extends out of the fluid storage bottle. The telescoping tube is configured to adjustably extend to any length out of the fluid storage bottle in order to reach different areas within a vaginal and anal cavity to provide a thorough flushing and cleansing of the vaginal and anal cavities.

### STATEMENT REGARDING FEDERALLY SPONSORED RESEARCH OR DEVELOPMENT

Not applicable.

### COPYRIGHT NOTICE

A portion of this disclosure contains material which is subject to copyright protection. The copyright owner has no objection to the photocopy reproduction by anyone of the patent document or the patent disclosure in exactly the form it appears in the Patent and Trademark Office patent file or records, but otherwise reserves all copyright rights whatsoever. 37 C.F. R 1.71(d).

### BACKGROUND OF THE INVENTIVE CONCEPT

### 1. Field of the Invention

Applicators are commonly used to retain cleansing fluids or solutions therein and to squeeze the cleansing fluid or solution out of a storage part thereof through a tube connected to the storage part in order to flush and cleans a vaginal or anal area of a human. The present inventive concept relates to a vaginal and anal applicator apparatus that is extendable and retractable to any desired length in order to be adjusted to reach any area within a vaginal or rectum desired to be flushed and cleansed.

### 2. Description of the Related Art

It is well known that vaginal and anal cavities can have poor sanitation due to menstrual or other personal bodily functions. However, flushing and cleansing a vaginal or anal cavity thoroughly can be difficult due to the challenges of reaching all areas within a vagina or anal cavity to properly cleanse them.

Many devices have been presented on the market to irrigate the vaginal area. U.S. Pat. No. 6,379,341 by Cho discloses a syringe for vaginal irrigation. This syringe includes an elastic barrel 51 containing a solution, an L-shaped elbow pipe 53 that is configured to engage with a threaded mouth of the barrel 51 and a hollow virginal insert 52 that engages with the elbow pipe 53 through a screw type engagement. Ejection holes 55 are formed along the insert 52.

However, this syringe is limited in its ability to reach all areas within a vaginal or anal cavity due to its limitation in length of the insert 52.

US Patent Publication No. 2006/0069338A1 by Bichsel et al. discloses a vaginal cleaning device 100 comprising an applicator 101, an applicator extension 102 and a handle 120. The applicator 101 comprises an axial support post 114 and a tubular applicator body 108. The extension 102 is non extendible, thus limiting the reach of the applicator 101 within a vaginal or anal cavity to fully cleanse the vaginal or anal cavity beyond the area in which the applicator 101 can reach.

CN 106421953A discloses a reusable vagina cleaning apparatus including a medicament reservoir 1, a medicine-introducing pipe 2 that extends into the reservoir 1, and a stopper 4. The stopper 4 stops the back end of the pipe 2 from extending out of the reservoir 1. Here the tip of the pipe 2 is includes a small flange 21 with holes 23 to allow fluid to exit the reservoir 1. However, the flange 21 with holes 23 is very small, thus limiting the area in which vagina can be cleansed. Further, this apparatus cannot provide any type of adjustment of the pipe 2 to reach different areas within a vaginal or anal cavity and can only reach areas equal to the length of the pipe 2.

Accordingly, there is a need for a vaginal and anal flushing and cleansing apparatus that can securely retain cleaning fluid in a leakproof manner while the apparatus is not in use.

There is also a need for a vaginal and anal flushing and cleansing apparatus that includes an extendable applicator tube which can adjust to any desire length to reach deep within a vaginal or anal cavity.

There is also a need for a vaginal and anal flushing and cleansing apparatus that includes an extendable applicator tube which can be locked at any desire length.

### SUMMARY OF THE INVENTIVE CONCEPT

The present general inventive concept relates to a vaginal and anal applicator that is extendable and retractable to any length in order to adjust to the area of a vaginal or rectum desired to be flushed and cleansed. More particularly, but not exclusively, the present inventive concept relates to a vaginal and anal applicator that includes an applicator tube with dispensing holes along a length thereof and is extendable and retractable to any length and can be locked at any desired length in order to adjust to the area of a vaginal or rectum desired to be flushed and cleansed.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a vaginal and anal applicator apparatus, comprising: an elastic squeezable bottle having a threaded neck extending outward from a first end thereof; a circular locking base having threads formed along an inner circumference thereof and configured to thread onto the threaded neck of the bottle, the circular locking base including: an internal tube having a first end extending from a first end thereof down pass the threads formed therein and including a hole formed through a second opposite end thereof to receive fluid from the elastic squeeze bottle, the internal tube being inserted into the elastic squeeze bottle when the circular locking base is threaded onto the threaded neck; and a threaded upper portion extending upward from the first end thereof; an applicator tube having a plurality of holes extending along a length thereof and configured to be slidingly insertable through the circular locking base and the internal tube and to receive fluid therethrough from the internal tube; and a threaded circular lock having threads formed along an inner circumference thereof to thread onto the threaded upper portion of the circular lock to lock the applicator tube in place when the threaded circular lock is fully threaded onto the threaded upper portion of the circular locking base.

In an example embodiment, the vaginal and anal applicator apparatus can further include a removable plug configured to be inserted into the hole formed through a second opposite end of the internal tube to block fluid within the elastic squeezable bottle from flowing into the internal tube.

In another example embodiment, the threaded upper portion of the circular locking base is configured to be compressible such that the threaded circular lock compresses the threaded upper portion of the circular locking base against the applicator tube when fully threaded onto the threaded upper portion of the circular locking base.

In another example embodiment, the circular locking base and internal tube combination, the applicator tube and the threaded circular lock are formed of a plastic.

The foregoing and/or other features and utilities of the present general inventive concept may also be achieved by providing a vaginal and anal applicator apparatus, comprising: an elastic squeezable bottle having a threaded neck extending outward from a first end thereof; a telescoping tube including a first tubular portion and a second tubular portion slidable within the first tubular portion, the first tubular portion including an circular locking base formed at a first end thereof including a threaded upper portion extending upward from the first end thereof and including threads formed therein to thread onto the threaded neck of the bottle to secure the first tubular portion within the elastic squeeze bottle and a second end with a hole extending therethrough to receive fluid from the elastic squeeze bottle, the second tubular portion including a plurality of holes extending along a length thereof and a hole through a first end extending into the first tubular portion to receive fluid from the first tubular portion; and a threaded circular lock having threads formed along an inner circumference thereof to thread onto the threaded upper portion of the circular locking base to lock the second tubular portion in place with respect to the first tubular portion when the threaded circular lock is fully threaded onto the threaded upper portion of the circular locking base.

In an example embodiment, the threaded upper portion of the circular locking base is configured to be compressible such that the threaded circular lock compresses the threaded upper portion of the circular locking base against the second tubular portion when fully threaded onto the threaded upper portion of the circular locking base.

In another example embodiment, the first tubular portion and the second tubular portion of the telescoping tube and the threaded circular lock are formed of a plastic material.

In another example embodiment, the vaginal and anal applicator apparatus can further include a removable plug configured to be inserted into the hole formed through the second end of the first tubular portion of the telescoping tube to block fluid from flowing from the elastic squeeze bottle into the first tubular potion.

Additional features and utilities of the present general inventive concept will be set forth in part in the description which follows and, in part, will be obvious from the description, or may be learned by practice of the general inventive concept.

### BRIEF DESCRIPTION OF THE DRAWINGS

These and/or other features and utilities of the present inventive concept will become apparent and more readily appreciated from the following description of the embodiments, taken in conjunction with the accompanying drawings of which:

FIG. 1 illustrates a vaginal and anal applicator apparatus in a fully extended mode, according to an example embodiment of the present inventive concept.

FIG. 1A illustrates a disassembled version of the vaginal and anal applicator apparatus 100 of FIG. 1.

FIG. 2 illustrates the vaginal and anal applicator apparatus as illustrated in FIG. 1 in a fully retracted mode, according to an example embodiment of the present inventive concept.

FIG. 3 illustrates the vaginal and anal applicator apparatus as illustrated in FIG. 1 in a fully extended mode, according to an example embodiment of the present inventive concept.

The drawing illustrates a process for making the invention according to an example embodiment of the present inventive concept and is not to be considered limiting in its scope, as the overall inventive concept may admit to other equally effective embodiments.

### DETAILED DESCRIPTION OF THE PREFERRED EMBODIMENTS

Reference will now be made in detail to the embodiments of the present general inventive concept, examples of which are illustrated in the accompanying drawings, wherein like reference numerals refer to the like elements throughout. The embodiments are described below in order to explain the present general inventive concept while referring to the figures. Also, while describing the present general inventive concept, detailed descriptions about related well-known functions or configurations that may diminish the clarity of the points of the present general inventive concept are omitted.

It will be understood that although the terms “first” and “second” are used herein to describe various elements, these elements should not be limited by these terms. These terms are only used to distinguish one element from another element. Thus, a first element could be termed a second element, and similarly, a second element may be termed a first element without departing from the teachings of this disclosure.

Expressions such as “at least one of,” when preceding a list of elements, modify the entire list of elements and do not modify the individual elements of the list.

All terms including descriptive or technical terms which are used herein should be construed as having meanings that are obvious to one of ordinary skill in the art. However, the terms may have different meanings according to the intention of the lexicographer, case precedents, or the appearance of new technologies. Also, some terms may be arbitrarily selected by the inventors, and in this case, the meaning of the selected terms will be described in detail in the detailed description herein. Thus, the terms used herein should be defined based on the generally defined meaning of the terms together with the description throughout this specification.

Hereinafter, one or more exemplary embodiments of the present general inventive concept will be described in detail with reference to accompanying drawings.

Example embodiments of the present general inventive concept are directed to a vaginal and anal applicator apparatus that is extendable and retractable to any desired length in order to be adjusted to reach any area within a vaginal or rectum desired to be flushed and cleansed.

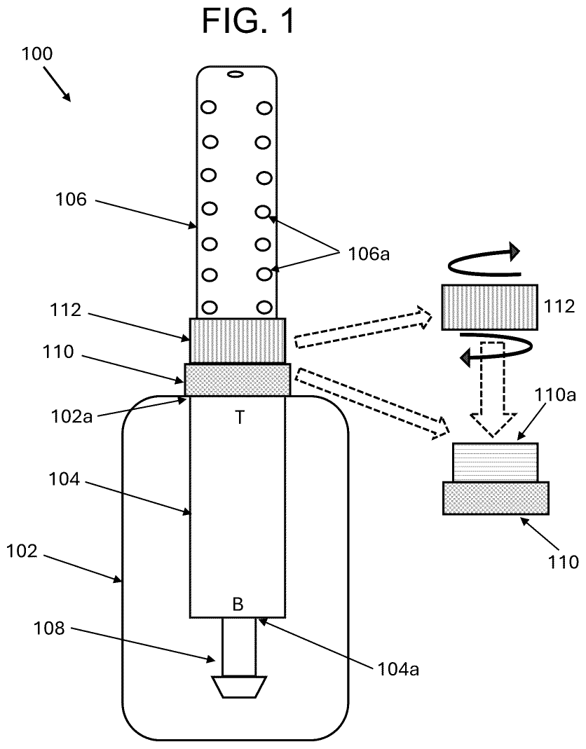

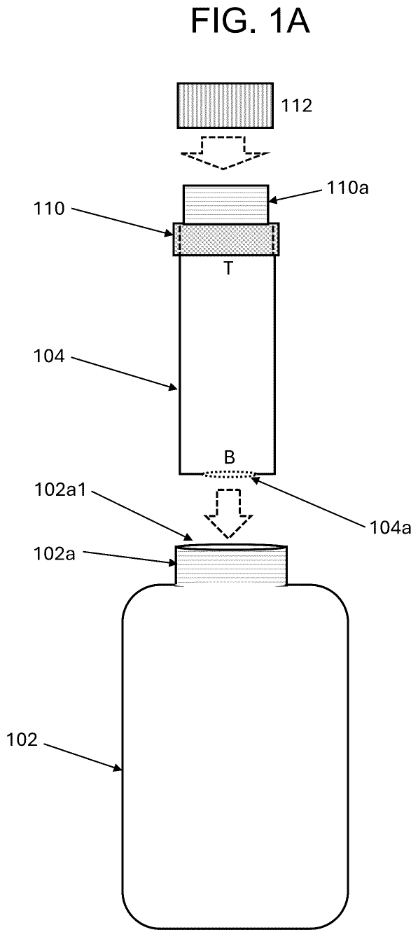

FIG. 1 illustrates a vaginal and anal applicator apparatus 100, according to an example embodiment of the present inventive concept. The vaginal and anal applicator apparatus 100 can include a squeeze bottle 102, which can be made of a squeezable material, such as plastic, or any equivalent type of material that can be flexibly squeezed and naturally return to its original shape.

FIG. 1A illustrates a disassembled version of the vaginal and anal applicator apparatus 100 of FIG. 1. Referring to FIGS. 1 and 1A, at a first upper end of the elastic squeeze bottle 102 can be formed a threaded neck 102a extending outward therefrom. The threaded neck 102a forms a hole 102a1 extending through the elastic squeeze bottle 102 in which a fluid (i.e., a cleaning solution) stored therein can exit the elastic squeeze bottle 102. The vaginal and anal applicator apparatus 100 can also include a locking base 110 having threads formed at an inner circumference thereof to be threaded onto the threaded neck 102a to securely attach the threaded locking base 110 to the elastic squeeze bottle 102. The locking base 110 can include an internal tube 104 connected thereto and extending downward from the locking base 110. The threaded locking base 110 can also include a compressible threaded upper portion 110a, which is described in more detail below. The locking base 110 and internal tube 104 can be formed as a single piece and can be formed of a plastic material or any other equivalent material that will perform the intended purposes as described herein.

The internal tube 104 is connected to the locking base 110 and therefore has an open top end T to enable fluid to flow through both the top end T of the internal tube 104 and the threaded locking base 110. The internal tube 104 can also include a fluid reception hole 104a formed through a bottom B portion thereof. The fluid reception hole 104a can be plugged shut with a funnel plug 108 when the vaginal and anal applicator apparatus 100 is in a storage mode. The funnel plug 108 is configured to prevent any fluid from entering into the internal tube 104 through the fluid reception hole 104a and exiting the bottle 102 when the funnel plug 108 is inserted into the fluid reception hole 104a. Alternatively, any equivalent stopper device can be used to close the fluid reception hole 104a without departing from the spirit and scope of the overall inventive concept. The internal tube 104 can be secured in place within the elastic squeezable bottle 102 when the threaded locking base 110 is fully threaded onto the threaded neck 102a of the bottle 102.

An extendable applicator tube 106 is configured to be insertable through the threaded locking base 110 and the top open end T of the internal tube 104. The extendable applicator tube 106 preferably includes a plurality of liquid dispense holes 106a formed therethrough along a length thereof. The extendable applicator tube 106 can be slid along the inner surface of the internal tube 104 until the extendable applicator tube 106 reaches the bottom end B of the internal tube 104. A threaded lock 112 can be slid over the extendable applicator tube 106 and can be threaded onto the threaded upper portion 110a of the threaded locking base 110 to lock the extendable applicator tube 106 at any position with respect to the threaded locking base 110 and internal tube 104. More specifically, the threaded lock 112 can be threaded partially onto the threaded upper portion 110a of the threaded locking base 110, at which point the extendable applicator tube 106 is free to be slid to any desired position with respect to the internal tube 104. Once the extendable applicator tube 106 is placed at the desired position the threaded lock 112 can be fully tightened onto the threaded locking base 110, which will compress the threaded upper portion 110a of the threaded locking base 110 against the extendable applicator tube 106 to secure the extendable applicator tube 106 in place at the desired position.

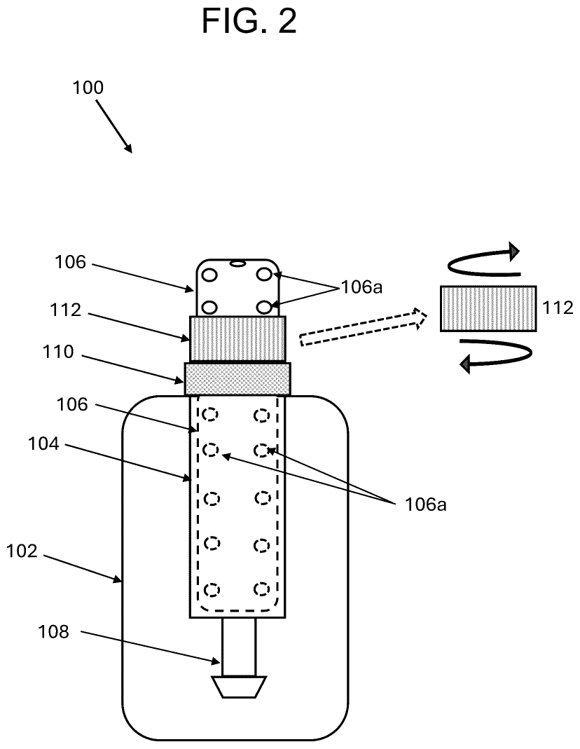

FIG. 2 illustrates the vaginal and anal applicator apparatus as illustrated in FIG. 1 in a fully retracted and closed mode, according to an example embodiment of the present inventive concept. Referring to FIG. 2, when the vaginal and anal applicator apparatus 100 is filled with a fluid, such as a cleansing solution, and ready to be stored for future use the funnel plug 108 will be inserted into the fluid reception hole 104a of the internal tube 104 and the extendable applicator tube 106 can be fully inserted into the internal tube 104 such that only a small upper end of the extendable applicator tube 106 extends out of the squeezable bottle 102. At this stage the threaded lock 112 can be fully tightened onto the threaded upper portion 110a of the threaded locking base 110 to compress the threaded upper portion 110a and secure the extendable applicator tube 106 at the position fully inserted into the internal tube 104 while the fluid reception hole 104a is plugged shut with the funnel plug 108, thus preventing any fluid with the bottle 102 from escaping.

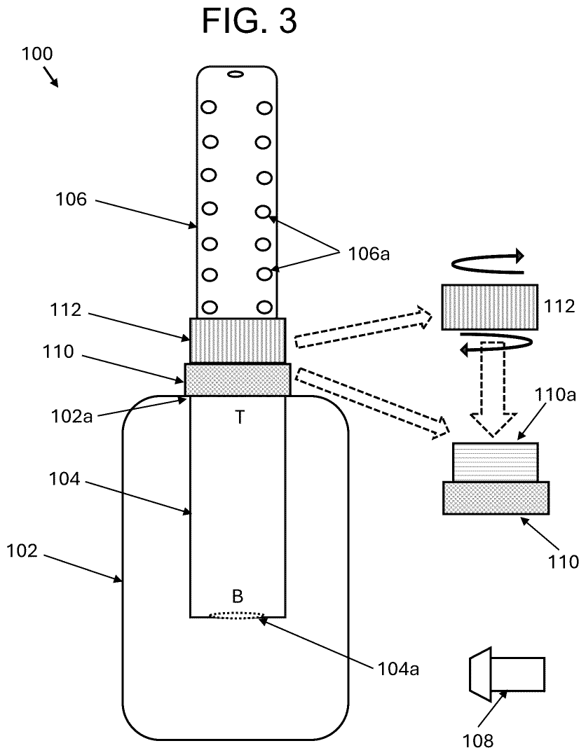

Referring to FIG. 3, when the vaginal and anal applicator apparatus 100 is ready to be used the threaded locking base 110 can be unscrewed from the threaded neck 102a of the bottle 102 to remove the internal tube 104 from the bottle 102. Once the internal tube 104 is removed from the elastic squeezable bottle 102 the funnel plug 108 can be removed from the fluid reception hole 104a of the internal tube 104. Once the funnel plug 108 is removed from the fluid reception hole 104a the internal tube 104 can be inserted back into the bottle 102 and the threaded locking base 110 can be threaded back onto the threaded neck 102a of the bottle 102.

Then the threaded lock 112 can be loosened with respect to the threaded upper portion 110a of the threaded locking base 110 so that the extendable applicator tube 106 will be free to be extended outward with respect to the squeezable bottle 102 to any desired length for proper application to a vaginal or anal cavity. Then by squeezing the elastic bottle 102 the fluid (i.e., cleansing solution) within the bottle 102 will be forced out through the internal tube 104 via the fluid reception hole 104a, through the extendable applicator tube 106 and out of the liquid dispense holes 106a formed through the extendable applicator tube 106.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A vaginal and anal applicator apparatus, comprising:
an elastic squeezable bottle having a threaded neck extending outward from a first end thereof;
a circular locking base having threads formed along an inner circumference thereof and configured to thread onto the threaded neck of the bottle, the circular locking base including:
an internal tube having a first end extending from a first end thereof down pass the threads formed therein and including a hole formed through a second opposite end thereof to receive fluid from the elastic squeeze bottle, the internal tube being inserted into the elastic squeeze bottle when the circular locking base is threaded onto the threaded neck; and
a threaded upper portion extending upward from the first end thereof;
an applicator tube having a plurality of holes extending along a length thereof and configured to be slidingly insertable through the circular locking base and the internal tube and to receive fluid therethrough from the internal tube; and
a threaded circular lock having threads formed along an inner circumference thereof to thread onto the threaded upper portion of the circular lock to lock the applicator tube in place when the threaded circular lock is fully threaded onto the threaded upper portion of the circular locking base.

2. The vaginal and anal applicator apparatus according to claim 1, further comprising:
a removable plug configured to be inserted into the hole formed through a second opposite end of the internal tube to block fluid within the elastic squeezable bottle from flowing into the internal tube.

3. The vaginal and anal applicator apparatus according to claim 1, wherein the threaded upper portion of the circular locking base is configured to be compressible such that the threaded circular lock compresses the threaded upper portion of the circular locking base against the applicator tube when fully threaded onto the threaded upper portion of the circular locking base.

4. The vaginal and anal applicator apparatus according to claim 1, wherein the circular locking base and internal tube combination, the applicator tube and the threaded circular lock are formed of a plastic.

5. A vaginal and anal applicator apparatus, comprising:
an elastic squeezable bottle having a threaded neck extending outward from a first end thereof;
a telescoping tube including a first tubular portion and a second tubular portion slidable within the first tubular portion, the first tubular portion including an circular locking base formed at a first end thereof including a threaded upper portion extending upward from the first end thereof and including threads formed therein to thread onto the threaded neck of the bottle to secure the first tubular portion within the elastic squeeze bottle and a second end with a hole extending therethrough to receive fluid from the elastic squeeze bottle, the second tubular portion including a plurality of holes extending along a length thereof and a hole through a first end extending into the first tubular portion to receive fluid from the first tubular portion; and
a threaded circular lock having threads formed along an inner circumference thereof to thread onto the threaded upper portion of the circular locking base to lock the second tubular portion in place with respect to the first tubular portion when the threaded circular lock is fully threaded onto the threaded upper portion of the circular locking base.

6. The vaginal and anal applicator apparatus according to claim 5, wherein the threaded upper portion of the circular locking base is configured to be compressible such that the threaded circular lock compresses the threaded upper portion of the circular locking base against the second tubular portion when fully threaded onto the threaded upper portion of the circular locking base.

7. The vaginal and anal applicator apparatus according to claim 6, wherein the first tubular portion and the second tubular portion of the telescoping tube and the threaded circular lock are formed of a plastic material.

8. The vaginal and anal applicator apparatus according to claim 5, further comprising:
a removable plug configured to be inserted into the hole formed through the second end of the first tubular portion of the telescoping tube to block fluid from flowing from the elastic squeeze bottle into the first tubular potion.
